(19) United States
(12) Patent Application Publication
(10) Pub. No.: US 2005/0050618 A1
(43) Pub. Date: Mar. 10, 2005

(54) Method and apparatus for providing pressure compensation to underwater goggles

(76) Inventor: Frank Carr Murphy, Austin, TX (US)

Correspondence Address: Joseph N. West Esq., Suite 200, 7501C North Capital of Texas Highway, Austin, TX, 78731, US

(21) Appl. No.: 10/933,558

(22) Filed: Sep. 4, 2004

Related U.S. Application Data
(60) Provisional application No. 60/500,554, filed on Sep. 5, 2003.

(51) Int. Cl. A61F 9/02

(52) U.S. Cl. 2/438

(57) ABSTRACT

The invention offers the methodology of providing continuous pressure compensation to dive masks and swim goggles through the use of flexible bladders or taps in the regulated air line, thus reducing the incidence of mask squeeze and thereby provide an adaptation of swim goggles for use in scuba diving. The methodology of supplying continuous pressure compensation also reduces the incidence of fogging in dive masks.

### CROSS-REFERENCE TO RELATED APPLICATIONS

Not Applicable

### STATEMENT REGARDING FEDERALLY SPONSORED RESEARCH OR DEVELOPMENT

Not Applicable

REFERENCE TO SEQUENCE LISTING, A TABLE OR A COMPUTER PROGRAM LISTING COMPACT DISC APPENDIX

Not Applicable

### BACKGROUND OF THE INVENTION

1. Field of the Invention

The present invention relates to a method and apparatus for providing pressure compensation to the space enclosed by underwater goggles or masks when properly worn on the face of the diver.

2. Description of the Prior Art

Apparatus for covering the eyes and other parts of the face when diving are not new to the art. Scuba diving masks have been around for years. Generally, these devices are used to protect the eyes by keeping water and water borne particles out of the eyes of the diver, and thereby assist with and improve the diver's vision during underwater diving activities. Typically, a dive mask encloses the eyes and nose of the diver in an air space framed by a soft rubber lining which serves to provide a tight contact between the mask and the diver's face.

Several inherent disadvantages exist with current day dive masks. First, dive masks generally create an environment which facilitates injury to the eyes and surrounding facial tissue under the mask caused by the pressure differential between the air inside the mask and the surrounding water pressure experienced during diving activities. This injury, commonly referred to as “ocular barotrauma” or “mask squeeze,” occurs because the air-filled space behind the mask is compressible, and when the surrounding water pressure increases unopposed during descent, a negative pressure develops inside the air-filled space, creating a painful suction on the eyes and surrounding facial tissue, thus crushing the mask into the diver's face. Typical ocular barotrauma, includes ocular lesions such as edema and ecchymosis of the eyelids, facial bruising and bleeding under the conjunctivae of the eyeballs.

During descent wearing a dive mask, divers must remember to periodically expel air through the nose into the mask in order to equalize the pressure inside the mask with that of the surrounding water and thus avoid the barotrauma. This practice, although eliminating or at least minimizing the barotrauma, has the inherent problem of causing the eye piece or lens of the mask to fog or cloud and thus restrict the diver's vision.

In addition to the ocular barotrauma and mask fogging, another disadvantage in many current day dive masks is the reduced peripheral vision of the diver caused by the soft rubber lining that typically frames the mask providing a suitable contact surface between the face and mask. Maximizing peripheral vision is a key to improved safety in diving activities.

Swim goggles, which generally cover only the eyes of the swimmer, are typically smaller than dive masks and provide far greater peripheral vision. Moreover, problems with fogging are reduced because the swim goggles do not cover the nose and therefore, gases exhaled from the nose do not enter the air space under the goggle eye piece. However, because such goggles do not typically cover the nose, pressure equalization of the air-filled space within the goggles is not possible. Therefore, such swim goggles are currently unsafe for diving purposes.

Accordingly, it would be desirable to provide a method for continuous pressure compensation of dive masks which reduces the incidence of fogging and which can be employed on swim type goggles, thus providing the diver with a safe alternative to dive masks and improved peripheral vision during diving activities.

### SUMMARY OF THE INVENTION

In view of the above, the inventor has developed the present invention in which the object is to offer a method of continuous pressure compensation for dive masks that reduces the incidence of fogging and with a preferred embodiment that employs a swim type goggle providing for improved peripheral vision.

### DESCRIPTION OF THE DRAWING

FIG. 1 is a view of conventional swim goggles with flexible tubes connected to the regulator mouthpiece and tapping the left and right eyepieces.

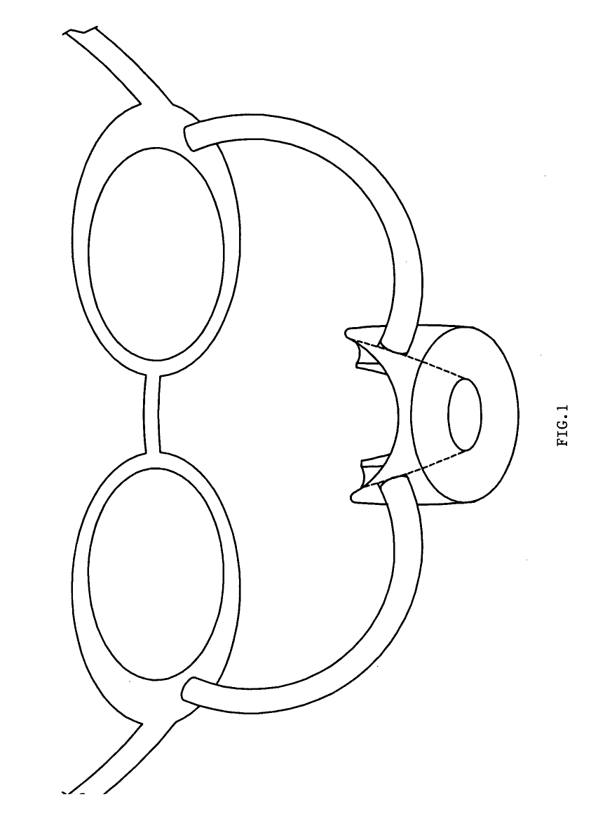

FIG. 2 is a view of conventional swim goggles with flexible air filled bladders tapping the left and right eyepieces.

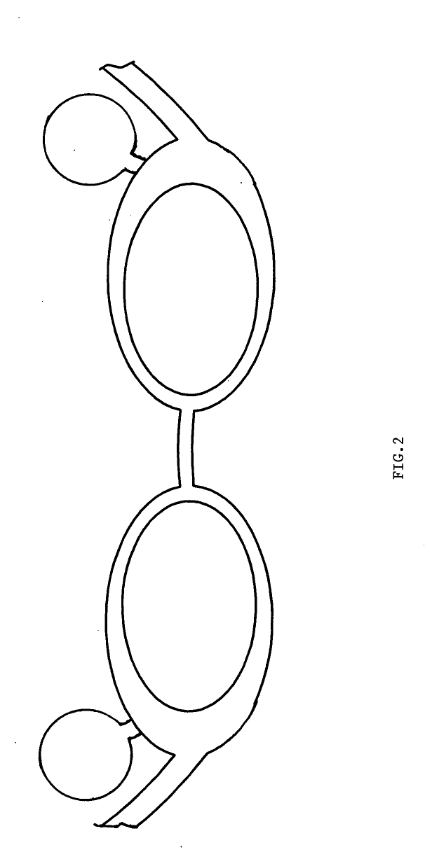

### DETAILED DESCRIPTION OF THE INVENTION

The present invention comprises a swim type goggle connected to the regulated air supply of a scuba unit using appropriate tubing or other connection means. Said connection means provide a continuous supply of regulated air to the air space between each eye piece of the swim goggle and the facial tissues and structures enclosed by each eye piece when said swim goggles are properly placed over the diver's eyes (the “Goggle Air Space”).

As typical swim goggles have a separate Goggle Air Space covering each eye with no cross-air flow between them, each Goggle Air Space (left and right) must have a separate connection with the regulated air (the “Left Connection Means” and the “Right Connection Means”). Said Left and Right Connection Means may tap the regulated air through attachment at a separate or singular point along the regulator mouth piece or at other appropriate locations along the regulated line. Said Left and Right Connection Means may tie into each associated Goggle Air Space at an appropriate location near or on each eyepiece.

An alternative embodiment of the present invention employs a typical dive mask in lieu of swim type goggles.

Another embodiment of the present invention employs the use of air filed bladders connected to the Goggle Air Spaces for swim type goggles and dive masks. Pressure compensation is achieved in this embodiment through contraction of the bladder as the diver descends.

While the invention has been described in what is considered the most practical and preferred embodiments, it is understood that the invention is not limited to the disclosed embodiments, but is intended to cover various modifications and similar arrangements and procedures within the spirit and scope of the broadest interpretation of the disclosed methods for providing pressure compensation to underwater goggles.

1. Goggles, having the air space(s) behind the goggle eyepiece(s) when properly worn by a diver, connected to a pressure compensation means.

2. The goggles in claim 1 with pressure compensation means comprising the divers regulated air stream.

3. The goggles in claim 1 with pressure compensation means comprising the divers regulated air stream and having connection means which taps the regulated air at the regulator mouthpiece and feeds the regulated air to the air space(s) behind the eyepiece(s) of the goggles, created when the goggles are properly worn on the face of the diver.

4. The goggles of claim 1 with pressure compensation means comprising the divers regulated air stream and with flexible tubing as the connection means tapping the regulated air at the regulator mouthpiece and feeding the regulated air through one tubing line to the air space behind the left goggle eyepiece and further feeding the regulated air through a second tubing line to the air space behind the right goggle eyepiece.

5. The goggles in claim 1 with pressure compensation means comprising air filled flexible bladder(s) connected to the air space(s) behind the goggle eyepiece(s) when properly worn by a diver.

6. The goggles of claim 1 with pressure compensation means comprising air filled flexible bladder(s) and wherein one flexible air filed bladder taps and supply's the air space behind the left goggle eyepiece and a second air filed bladder taps and supply's the air space behind the right goggle eyepiece.
